(19) United States
(12) Patent Application Publication
(10) Pub. No.: US 2018/0254093 A1
(43) Pub. Date: Sep. 6, 2018

(54) CRYPTOGRAPHICALLY SECURE MEDICAL TEST DATA DISTRIBUTION SYSTEM USING SMART TESTING/DIAGNOSTIC DEVICES

(71) Applicant: Allocrypt Inc., La Jolla, CA (US)

(72) Inventor: Gregory G. Rose, La Jolla, CA (US)

(21) Appl. No.: 15/911,027

(22) Filed: Mar. 2, 2018

Related U.S. Application Data
(60) Provisional application No. 62/466,338, filed on Mar. 2, 2017.
(60) Provisional application No. 62/623,008, filed on Jan. 29, 2018.

(51) Int. Cl. G16H 10/40; G16H 15/00; H04L 29/06; G06F 3/12

(52) U.S. Cl. CPC G16H 10/40; G06F 3/1238; H04L 63/0478; G16H 15/00

(57) ABSTRACT

A diagnostic device is provided that obtains test results and transmits a cryptographically secure version of such test results. The diagnostic device may be provisioned with a unique first public key and first private key pair and a unique device identifier for the diagnostic device. A link may be established with a mobile communication device associated with a unique patient identifier. A test request may be received from the mobile communication device including the patient identifier. The diagnostic device may then verify whether the requested test can or should be performed based, at least partially, on the patient identifier. If the patient identifier is verified, the diagnostic device may perform the requested test to obtain a test result. The test result may be encrypt/signed using the first private key and a first authorized receiver public key to obtain a first encrypted result that is transmitted to the mobile communication device.

### BACKGROUND

The present Application for Patent claims priority to U.S. Provisional Application No. 62/466,338 filed Mar. 2, 2017, and U.S. Provisional Application No. 62/623,008 filed Jan. 29, 2018, both of which are hereby expressly incorporated by reference.

### FIELD

Various features relate to the methods and devices for securely testing and sharing medical data, and more particularly to the use of smart medical/drug monitoring and/or diagnosis devices that test blood and/or bodily fluids and securely provide test results to patients, medical service providers as well as anonymously provide test results to a third parties.

### BACKGROUND

Medical testing/diagnostic devices are increasingly used drug monitoring and/or test patient's blood and/or bodily fluids. However, the results for such drug/medical tests must be securely provided to the patient and/or medical service providers.

Therefore, a way is needed to securely and conveniently share test data/results from the testing/diagnostic devices with patients and/or medical service providers.

### SUMMARY

A first aspect provides a diagnostic device comprising a storage device, a testing sensor device, a communication interface, and a processing circuit. The storage device for storing a unique first public key and first private key pair and a unique device identifier for the diagnostic device. The testing sensor device may be configured to receive blood or bodily fluids and providing a test result. The communication interface for transmitting information to/from the diagnostic device. The processing circuit configured to: (a) establish a communication link with a mobile communication device, where the mobile communication device is associated with a unique patient identifier; (b) receive a test request from the mobile communication device including the patient identifier; (c) verify whether the requested test can be performed based, at least partially, on the patient identifier; (d) perform, if the patient identifier is verified, the requested test to obtain a test result; (e) encrypt and/or sign the test result using the first private key and a first authorized receiver public key to obtain a first encrypted result; (f) encrypt and/or sign the test result using the first private key and a second authorized receiver public key to obtain a second encrypted result; and/or (g) send the first encrypted result and second encrypted result to the mobile communication device.

The diagnostic device may be pre-configured with a stored patient identifier, and verifying whether the requested test can be performed includes comparing the stored patient identifier and the patient identifier received with the test request.

The diagnostic device may be a one-time-use device and is pre-configured to work only if the test is requested with the unique patient identifier.

The test request further may include a diagnostic device identifier, and the method further comprises: confirming whether the unique device identifier matches the diagnostic device identifier prior to proceeding with the test request.

A second aspect provides a method operational on a mobile communication device, comprising: (a) obtaining a unique patient identifier associated with a user of the mobile communication device; (b) establishing a short-range wireless link with a diagnostic device; (c) sending a test request to the diagnostic device including the patient identifier; (d) receiving a first encrypted test result that is encrypted with a first public key associated with the diagnostic device; (e) receiving a second encrypted test result that is encrypted with a second public key associated with a printer device of an authorized receiver of the test result; (f) forwarding the first encrypted result and second encrypted result to a centralized authentication server, (g) receiving a confirmation that the second encrypted result has been delivered to a registered service provider, and/or (h) displaying the test result on the mobile communication device.

A third aspect provides a method for performing cryptographically secure medical testing and distribution of test results, comprising: (a) enrolling a medical service provider with a centralized service, wherein a unique printer box is co-located with the medical service provider and has an associated printer identifier, and a printer public key and printer private key pair; (b) enrolling a patient with the centralized service, wherein a mobile communication device for the patient has an associated patient identifier, and patient public key and patient private key pair; (c) distributing a diagnostic device that has a diagnostic device public key and a diagnostic device private key pair, and the diagnostic device is pre-configured to operate only with the patient by communicating with mobile communication device and cryptographically authenticating the patient based on, at least, the patient identifier; (d) performing a test at the diagnostic device to obtain a test result; (e) encrypting the test result using at least one of the patient public key or the printer public key; (f) signing the encrypted test result using the diagnostic device private key; (g) distributing the signed and encrypted test result to the mobile communication device; (h) forwarding, either directly or indirectly, the signed and encrypted test result to the printer device; (i) authenticating the signed and encrypted test result using the pre-distributed diagnostic device public key; and/or (j) decrypting the encrypted test result to obtain the test result using the printer device private key.

A fourth aspect provides a diagnostic device comprising: a testing sensor, a communication interface, and a processing circuit. The testing sensor configured to receive blood or bodily fluids and providing a test result. The communication interface adapted for transmitting information to/from the diagnostic device. The processing circuit configured to: (a) establish a communication link with a mobile communication device; (b) receive a test request from the mobile communication device; (c) perform the requested test to obtain test results; (d) remove patient-identifying information from the test results; (e) encrypt the test results using the first public key for a third party to obtain encrypted test results; (f) send the encrypted test results to the mobile communication device; (g) generate a zero-knowledge proof of the test results and a diagnostic device secret key; (h) encrypt the zero-knowledge proof to obtain an encrypted zero-knowledge proof; and/or (i) send the encrypted zero-knowledge proof along with the encrypted test results encrypted result to the mobile communication device. The encrypted test results may be transmitted to the third party via a relay system that removes device and/or network identifying information prior to forwarding the encrypted test results.

### BRIEF DESCRIPTION OF THE DRAWINGS

Various features, nature and advantages may become apparent from the detailed description set forth below when taken in conjunction with the drawings in which like reference characters identify correspondingly throughout.

FIG. 1 (comprising FIGS. 1A, 1B, 1C, and 1D) illustrates a first example of a system for using a disposable smart diagnostic/testing device to test bodily fluids (e.g., blood, to monitor for the presence of a particular drug, etc.) and securely distribute test results to patients and/or medical service providers.

FIG. 2 (comprising FIGS. 2A, 2B, 2C, 2D, and 2E) illustrates a second example of a system for using a reusable diagnostic/testing device to test bodily fluids, functions, and/or conditions (e.g., blood, to monitor for the presence of a particular drug, etc.) and securely distribute test results to patients and/or medical service providers, and anonymously distribute test results to third parties.

FIG. 3 illustrates an exemplary diagnostic device configured to test one or more bodily fluids or biological samples and securely provide test results.

FIG. 4 illustrates a method operational in a diagnostic device to securely share test results.

FIG. 5 illustrates an exemplary printer device configured to securely provide test results.

FIG. 6 illustrates a method operational by a mobile communication device to facilitate performing a test with a diagnostic device and securely share test results.

FIG. 7 illustrates an exemplary mobile communication device configured to test one or more bodily fluids or biological samples and securely provide test results.

FIG. 8 illustrates a method operational by a mobile communication device to facilitate performing a test with a diagnostic device and securely share test results.

### DETAILED DESCRIPTION

In the following description, specific details are given to provide a thorough understanding of the various aspects of the disclosure. However, it will be understood by one of ordinary skill in the art that the aspects may be practiced without these specific details. For example, circuits may be shown in block diagrams in order to avoid obscuring the aspects in unnecessary detail. In other instances, well-known circuits, structures and techniques may not be shown in detail in order not to obscure the aspects of the disclosure.

The word “exemplary” is used herein to mean “serving as an example, instance, or illustration.” Any implementation or aspect described herein as “exemplary” is not necessarily to be construed as preferred or advantageous over other aspects of the disclosure. Likewise, the term “aspects” does not require that all aspects of the disclosure include the discussed feature, advantage or mode of operation.

### Overview

One aspect provides a security threat model and architecture for a drug monitoring service and/or blood or bodily fluid monitoring service that handles communication for a disposable diagnostic/testing device, as well as “point-of-care” diagnostic/testing devices. For instance, these diagnostic/testing devices may inexpensively and easily detect levels of certain medications in the blood. Accurate measurement of these levels enables better medical outcomes and significantly less expense for the same effective treatment. One example medication is Tysabri™.

In some implementations, the disposable diagnostic/testing device may be delivered to a patient for a single use, or a limited number of uses for the same patient. The detected level of medication must be securely conveyed to the treating medical professional.

In other implementations, diagnostic/testing device may be reusable, e.g., installed in a medical office, but not necessarily that of the patient's treating professional.

According to one aspect, the patient is assumed to have a personal smart phone/tablet device with an appropriate application installed therein and capable of communicating over a network. This phone/table device may be used to facilitate a secure connection between the testing device and the treating professional.

The treating professional may also have another specialized output device, e.g., an Internet-connected specialized printer, so that test/diagnostic results can be securely received almost instantly after the test is done.

### Exemplary Threat Model

Confidentiality: In one example, the detected level of medication, and which medication is being tested for, should not be revealed to any party other than the patient and a medical service provider (e.g., doctor, physician, nurse, etc.). It should also be observed that, initially, when there is only one medication being tested, the mere presence of the communication may reveal which medication it is. Once more than one medication is being tested, nothing in the messaging should allow a third party to distinguish between the possible alternatives.

Authentication: The phone/tablet device may be registered to a particular patient, and represents the patient's identity to the measuring testing device. When the patient interacts with the service provider, at some point, the patient identifies the service provider (and the associated printer device). The patient's phone/tablet device can then present the patient's information to the testing device, implicitly authenticating and authorizing the service provider as a valid recipient of the test results.

Integrity: It should not be possible for any third party to alter the output/results of the testing device (e.g., a measuring box) without being detected, or to create an apparently valid measurement or test result. This requirement implies that the testing devices are themselves identifiable, so that they can digitally sign measurements. Similarly the print devices should be identifiable so that they can be targets of the communication (e.g., they can securely receive the test results).

### Exemplary Infrastructure

A centralized service may enable the confidential/secure distribution of test results from testing devices. In one example, this service may be a cloud-hosted service to provide scalability. This service may be responsible for:
Registering users (e.g., patients, their representatives, and health professionals) in the system.
Registering devices (phones, measuring/diagnostic devices and printing devices). This might include provisioning such devices with cryptographic keys at manufacturing time.
Acting as a forwarding service, receiving test results and forwarding them to the appropriate print device. This might include backup and archiving functions, and buffering in case a particular print device is offline, or the patient changes health professional.
At a cryptographic level it will need to act like a private Certificate Authority.
It will implement various statistical and audit functions.

### Exemplary Testing Devices (Diagnostic/Testing Boxes)

The diagnostic/testing boxes/devices described herein may include an integrated lab that permits testing blood/bodily fluid for various conditions, including the presence and/or level of one or more drugs. Such diagnostic/testing boxes/devices may have an integrated power source (e.g., battery, power cell, etc.) or may be externally powered. Additionally, the diagnostic/testing boxes/devices may include one or more circuits and/or processing circuits configured to test for drug levels in blood or bodily fluids and/or compute a result. The boxes/devices may also include a communication interface/circuit (wired/wireless) to communicate with other devices. In various examples, the communication interface/circuit may a Bluetooth-compatible interface or internet-capable communication interface.

All devices in the system may know a root public key of the centralized service (e.g., part of a root public and private key pair), and are capable of verifying messages and certificates that chain up to that root key. In one example, the formats of the data in the certificates may be compatible with (a subset of) the X.509 standard, but it is not intended that these certificates will be part of the normal certificate authority (CA) infrastructure.

The diagnostic/testing devices may be provisioned with a unique identifier (e.g., a serial number) and a unique public/private key pair. These keys will be known to the infrastructure centralized service for backup purposes. The unique identifier and public key will also be printed on the testing device, e.g., an optically readable format such as 2D barcode or in other ways. The infrastructure centralized service may also issue a certificate to be stored in the testing device (and maybe on the barcode depending on capacity).

In some implementations, the diagnostic/testing devices may be a disposable (e.g., one-time use) devices, which are intended to be shipped to a particular patient, might be pre-provisioned with the intended patient's certificate, making the device useless if stolen.

In other implementations, the diagnostic/testing devices may be reusable devices, and may be ship to, for instance, medical services providers.

The diagnostic/testing devices may be adapted to perform on-board testing of blood or bodily fluids and provide results of such tests. In various implementations, the diagnostic/testing devices may use a wired or wireless interface to communicate the test results to the patient, e.g., to the patient's phone/tablet device (e.g., using a Bluetooth link, etc.).

### Exemplary Printer Devices

Another aspect provides printer devices that may be distributed to medical service providers. The printer boxes/devices described herein may include an integrated power source (e.g., battery, power cell, etc.) or may be externally powered. Additionally, the printer boxes/devices may include one or more circuits and/or processing circuits configured to print/output and/or store test result received by the printer boxes/devices. The boxes/devices may include a communication interface/circuit (wired/wireless) to communicate with other devices. In various examples, the communication interface/circuit may a Bluetooth-compatible interface or internet-capable communication interface. The boxes/devices may be able to directly interface to the health provider's medical records system.

The printer devices may be connected, e.g., via either WiFi or a wired connection, to the Internet (or a communication network). The printer devices may serve to print out reports, which can then be transcribed into the medical service provider's patient records system, thereby obviating the need for separate certification of these testing devices, and avoiding the complexity of having to be compatible with a number of different medical records systems. Alternative implementations may provide interfaces for the diagnostic/testing devices to communicate directly with such medical records systems.

### Exemplary Enrollment of Medical Service Providers

A medical service provider may purchase or obtain the printer device from the centralized service, and go online from the medical service provider's office to provide various registration information. The centralized service may then issue certificates for the printer device, including cryptographic keys within the printer device. In some implementations, the medical service provider may perform a registration or initialization process from a phone that can provide the centralized service with location information.

The patient may go to see the medical service provider at some point and will receive instructions to download and install an application on the patient's phone/tablet device in advance of this actual visit. The application will gather various registration information from the patient, and issue the patient's certificates to be stored on the patient's phone/tablet.

At the medical service provider's office, as well as the usual medical stuff, the patient can start/execute the application, and specify that the medical service provider is the intended medical professional for delivery of the test results. The patient's location at this time might be used to associate the patient with the medical service provider automatically. The patient could also use the application and phone's/tablet's camera to scan a barcode on the medical service provider's printer device to establish this association.

### First Exemplary Approach to Cryptographically Securing Test Results (Disposable Testing/Diagnostic Device/Box)

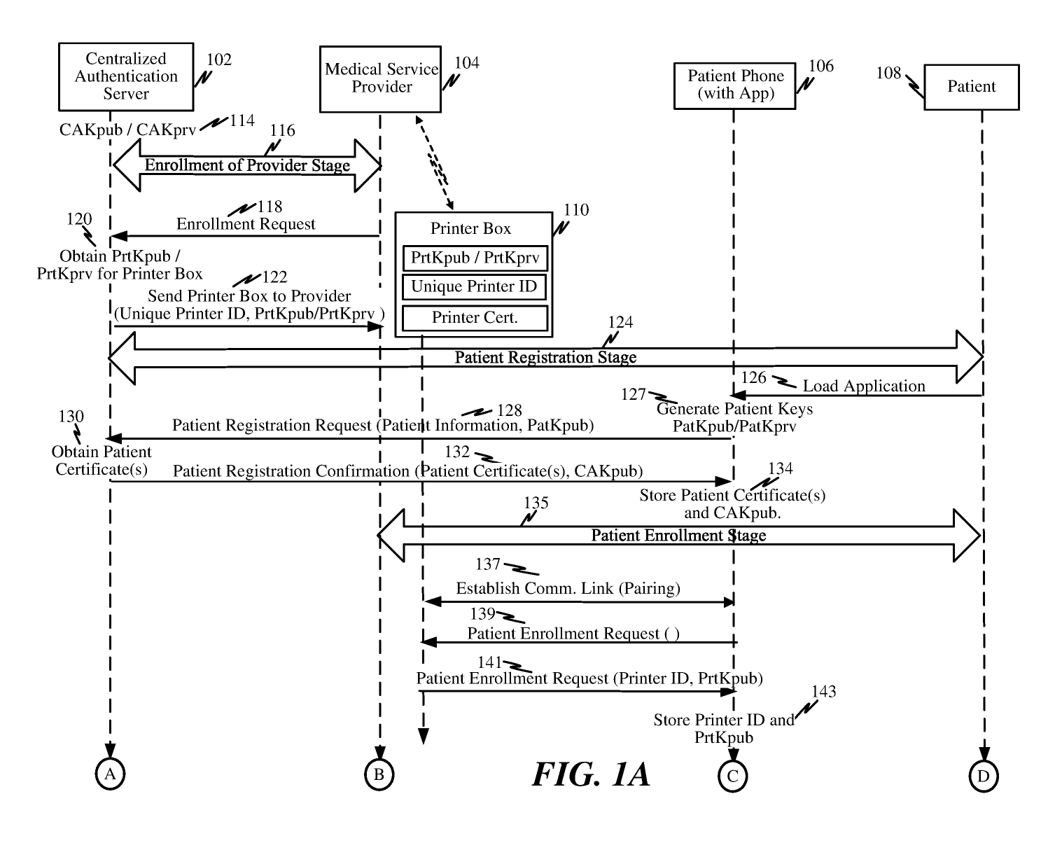

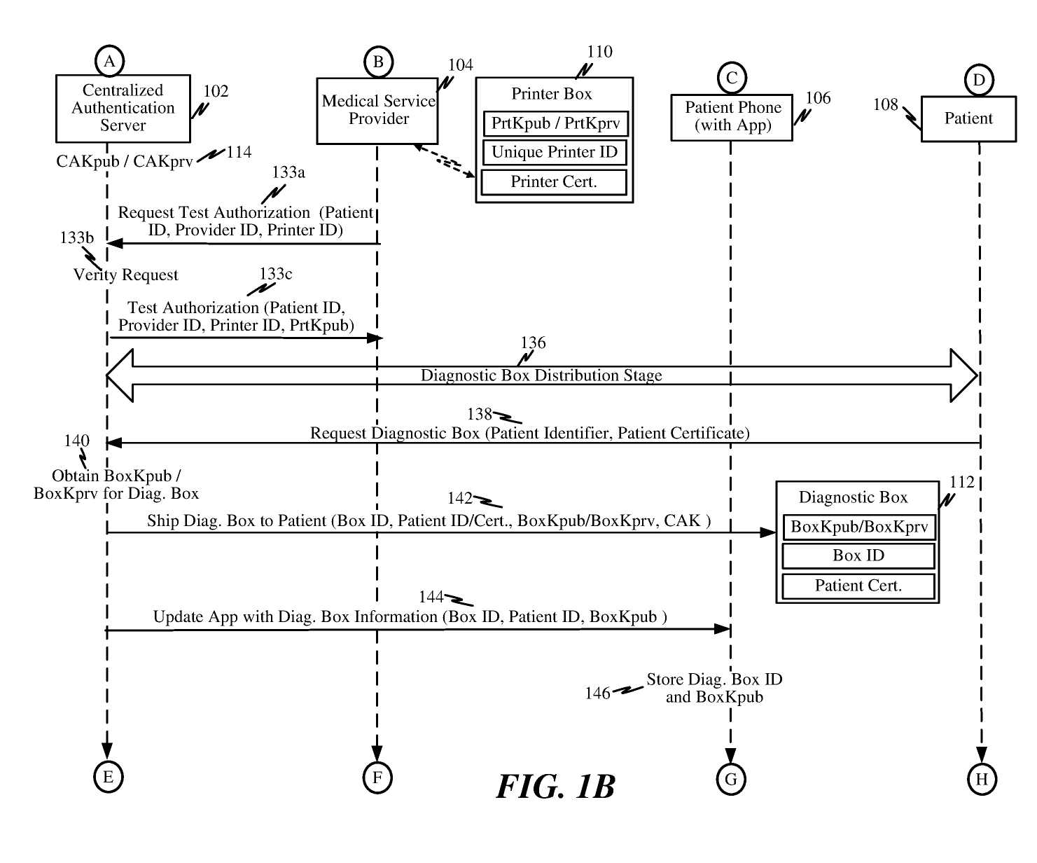

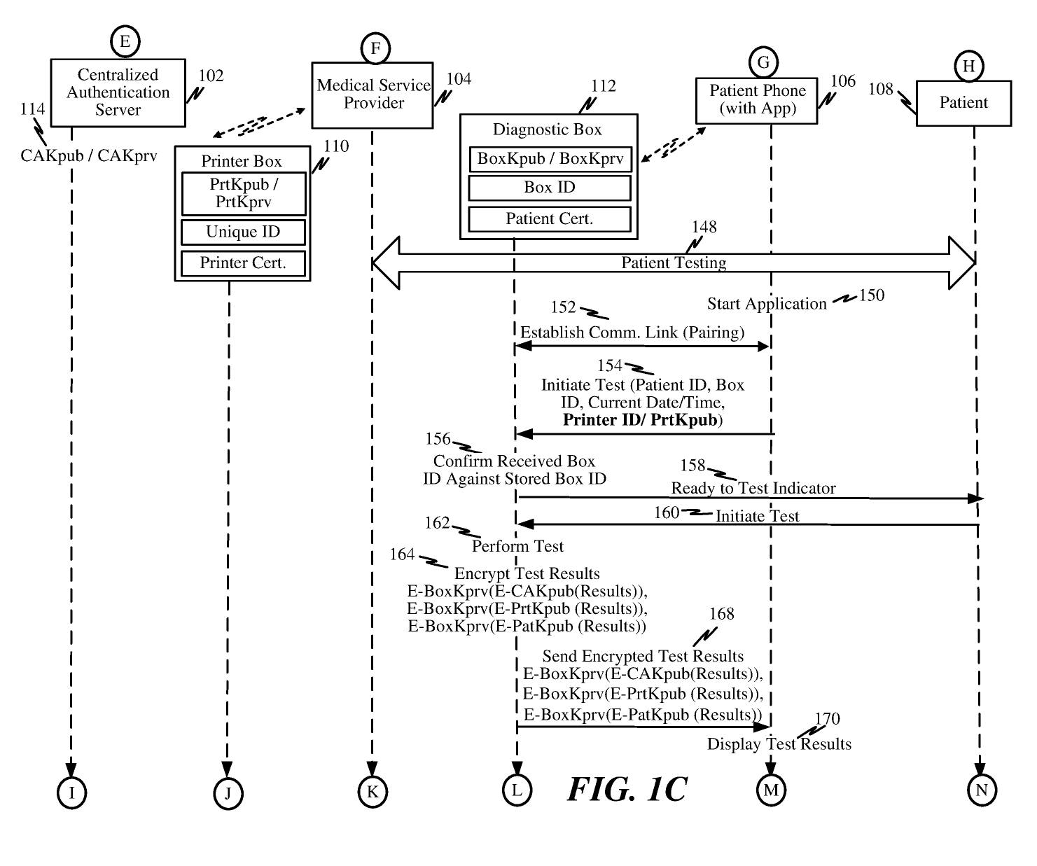

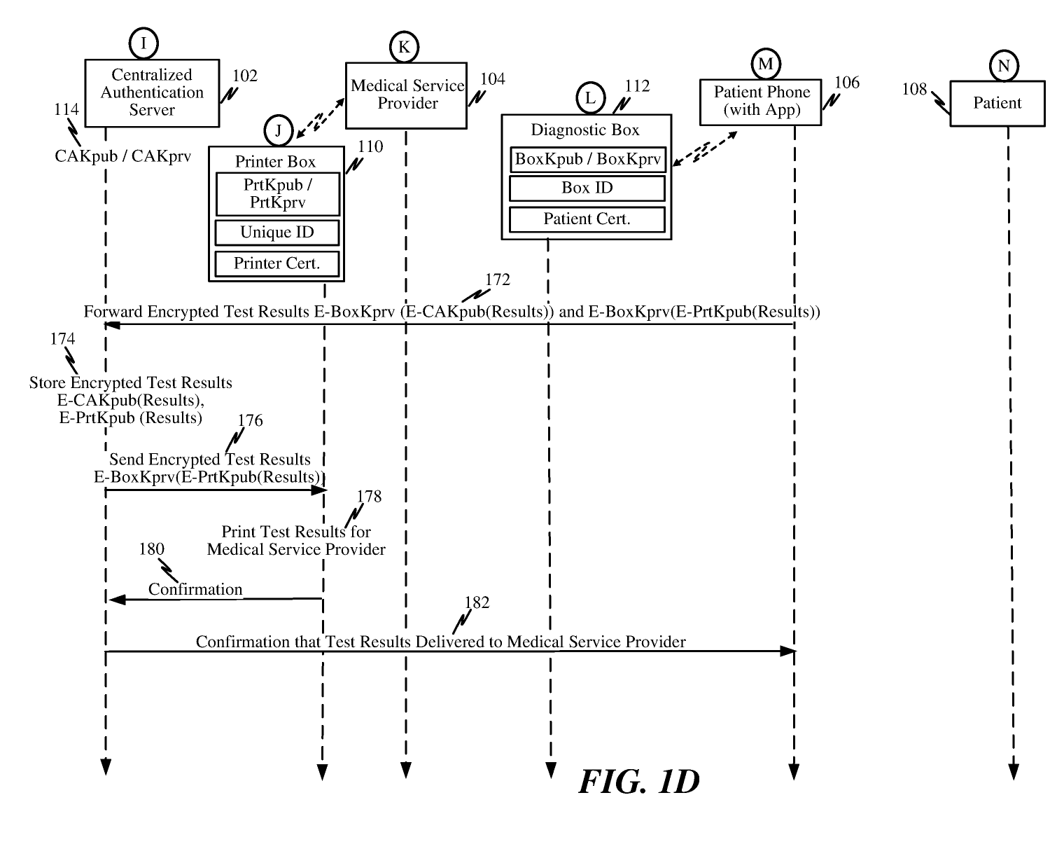

FIG. 1 (comprising FIGS. 1A, 1B, 1C, and 1D) illustrates a first example of a system for using a disposable diagnostics/testing device to test bodily fluids (e.g., blood, to monitor for the presence of a particular drug, etc.) and securely distribute test results to patients and/or medical service providers. This system comprises a centralized authentication server 102 (e.g., centralized service), a medical service provider 104, a printer box/device 110, a patient 108, a patient phone 106 (e.g., patient communication device), and/or a disposable (one-time-use) diagnostic/testing box/device 112. A certificate authority public CAKpub and private CAKpry key pair may be defined. The public key CAKpub may be shared with other parties so as to secure (e.g., verify digitally signed communications from the centralized authentication server, and to encrypt messages to the centralized server).

During an enrollment of provider stage 116, the medical service provider 104 may request to enroll 118 with the centralized authentication server 102 (e.g., or the centralized service). In response to the enrollment request 118, the centralized authentication server 102 may obtain/generate a patient-specific public PrtKpub and private PrtKpry key pair and associates it with a printer box/device 110 that is sent to the medical service provider 104. The printer box/device 110 may also have a unique printer identifier and/or a printer certificate. In one example, the printer certificate may include the printer public key PrtKpub, which may be specific to each printer box/device and may be provisioned by the centralized authentication server 102 in advance, and a digital signature of that public key by the centralized authentication server private key (CAKpry key). In this manner, the printer public key PrtKpub, the printer private key PrtKpry the unique printer identifier, and/or a printer certificate may be securely sent 122 within the printer box/device 110.

At a patient registration stage 124, the patient 108 may load a software application 126 into the patient phone 106 (e.g., or other personal communication device such as a tablet). Upon attempting an initial registration the software application may generate or obtain a patient public/private key pair PatKpub/PatKpry 127. The software application then sends a patient registration request 128 to the centralized authentication server 102, where the patient registration request 128 may include patient information (e.g., name, address, a patient identifier, etc.) and the patient public key PatKpub. The centralized authentication server 102 may then obtain patient certificates 130 and sends a patient registration confirmation 132, along with the patient certificates 130, to the patient phone 106. The certificate authority public key CAKpub may also be sent to the patient phone 106 to permit it to verify signed communications from, and securely encrypt communications to, the centralized authentication server 102. The patient certificates and certificate authority public key CAKpub may then be stored at the patient phone 106.

During a patient enrollment stage 135, the patient 108 may visit the medical service provider 104 and performs a patient enrollment procedure in which the patient's phone 106 establishes a communication link 137 with the printer box 110. The patient's phone 106 then sends a patient enrollment request 139 to the printer box 110. In reply, the patient's phone 106 (or software application therein) receives the printer identifier (Printer ID) and/or the printer public key PrtKpub 141, which it can subsequently use to send test results from the diagnostic box 112 to the printer box 110.

In one exemplary implementation, the medical service provider 104 may request a test authorization 133a from the centralized authentication server 102. The centralized authentication server 102 verifies the request 133b, and provides a test authorization 133c to the medical service provider 104.

In a diagnostic box distribution stage 136, the patient 108 may start the software application within the patient phone 106. The patient phone 106 may contact the centralized authentication server 102 (e.g., via the Internet and using a TLS connection authenticated with the patient certificate). In some implementations, the patient 108 may utilize the software application to request a diagnostic box 138, where the request includes the patient identifier and the patient certificate. The centralized authentication server 102 may obtain/generate a unique public and private key pair BoxKpub/BoxKpriv 140 for a diagnostic box and ships the diagnostic box 142 with a unique box identifier Box ID, the patient identifier or certificate, and the key pair BoxKpub/BoxKpry therein. The centralized authentication server 102 may also send an update 144 with the diagnostic box information (e.g., box identifier, patient identifier, box public key BoxKpub) to the patient phone 106. The software application in the patient phone 106 may then store 146 the diagnostic box identifier and the diagnostic box/device public key BoxKpub.

For patients that require periodic testing, a new disposable diagnostic box may be shipped to the patient 108 periodically. Each such diagnostic box may be preconfigured with the patient's certificate so that the diagnostic box 112 will not work for anyone who does not have access to the patient phone 106. Alternatively the diagnostic box may be capable of performing a limited number of repeat tests.

During a testing stage 148, the patient 108 may perform medical tests using the diagnostic box 112. In one example, the patient 108 may visit a doctor (medical service provider 104) to establish a link between the patient's phone 106 and the correct doctor's printer 110. Once established, the patient 108 may perform the test at the medical service provider location or elsewhere (e.g., at a remote location, the patient's home, etc.). Since the software application knows the correct printer box 110 identifier, the diagnostic box results can be sent by the patient phone 106 to the correct doctor (medical service provider 104). The patient phone 106 and the diagnostic box 112 may be paired (e.g., using Bluetooth) to establish a communication link 152. In some implementations, no security may be needed on this link because the security may be done at a higher protocol layer.

The patient 108 starts the software application 150 in the patient phone 106 which may then initiate a test 154 with the diagnostic box 112 by sending the patient identifier, the box identifier, a current date/time, the printer identifier, and/or the printer public key PrtKpub. The diagnostic box 112 uses the received box identifier and stored box identifier to confirm 156 whether the patient should be permitted to take the test using the diagnostic box 112. If the received and stored box identifiers match, the diagnostic box 112 may send an indicator 158 that the diagnostic box is ready to perform a test. The patient 108 may then initiate the test 160 (e.g., provide a blood/fluid sample). The diagnostic box 112 may then perform the test 162.

When the test result is available, two copies of the result are encrypted 164 by the diagnostic box 112 and sent 168 to the patient phone 106. In one example, the test results may be encrypted using the certificate authority public key CAKpub to produce a first encrypted result E-CAKpub(Results). In another example, the test results may also be encrypted using the printer box public key PrtKpub to produce a second encrypted result E-PrtKpub(Results). In some implementations, the test results may be further signed using the diagnostic box private key BoxKpry so that a receiving party/entity can be certain that the test was performed by the diagnostic box 112 (i.e., by using the diagnostic box public key BoxKpub to verify). The first and second encrypted results E-BoxKprv(E-CAKpub(Results)) and E-BoxKprv(E-PrtKpub(Results)) are sent 168 to the patient phone 106. According to an optional aspect, the test results may also be sent to the patient phone 106, for instance, by encrypting the results using the patient public key PatKpub and then diagnostic box private key BoxKpry to obtain a third encrypted result. This third encrypted results may be verified or decoded using the diagnostic box public key BoxKpub and the patient private key PatKpry and the results may be displayed 170 by the software application on the patient device 106.

The results first and second encrypted results E-BoxKprv(E-CAKpub(Results)) and E-BoxKprv(E-PrtKpub(Results)) are both forwarded 172 to the centralized authentication server 102 by the patient phone 106. The centralized authentication server 102 may store 174 both encrypted results for backup and potential legal purposes. The second encrypted result E-BoxKprv(E-PrtKpub(Results)) may be delivered/sent 176 to the printer box 110 from where the medical service provider 104 can print and/or store them in a medical records system. The printer box 110 can verify the signature on the result to confirm that the test was performed by a specific diagnostic box. That is, the printer box 110 may use the diagnostic box public key BoxKpub and the printer box private key PrtKpry to decrypt and verify the encrypted results.

The printer box 110 may send a confirmation reply 180 upon receipt of the encrypted test results E-PrtKpub(Results). In response, the centralized authentication server 102 may send a confirmation 182, to the patient phone 106, that the test results have been delivered to the medical service provider 102. This informs the patient 108 that medical service provider 104 has received the test results.

All messages within this system may be encrypted with an intended recipient's public key, and authenticated (e.g., verified or decrypted) with the intended sender's private key so that the recipient can use the sender's public key to verify authenticity. As is well known in the industry, encryption and signing operations might use different but associated keys, but we describe them as if it is a single key pair for both operations.

It is contemplated that the various steps, stages, and/or operations described herein may be combined, rearranged, and/or modified without departing from the novel aspects described.

### Second Exemplary Approach to Cryptographically Securing Test Results (Reusable Testing/Diagnostic Device/Box)

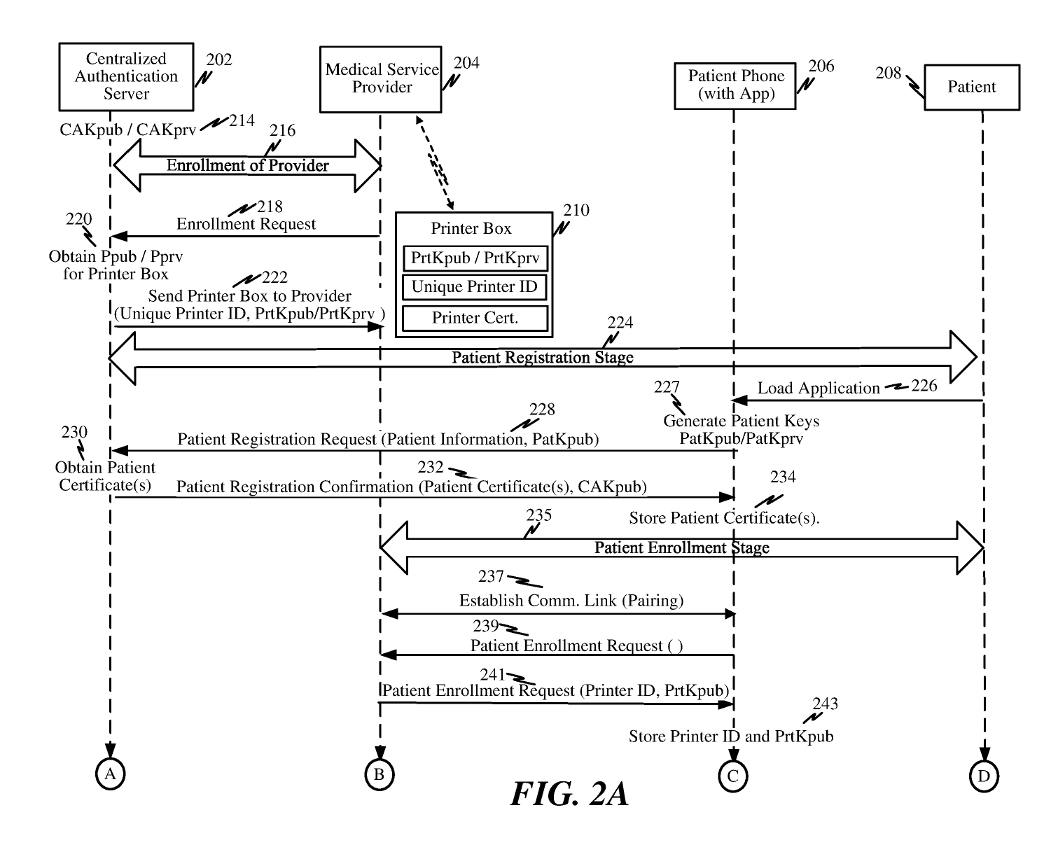

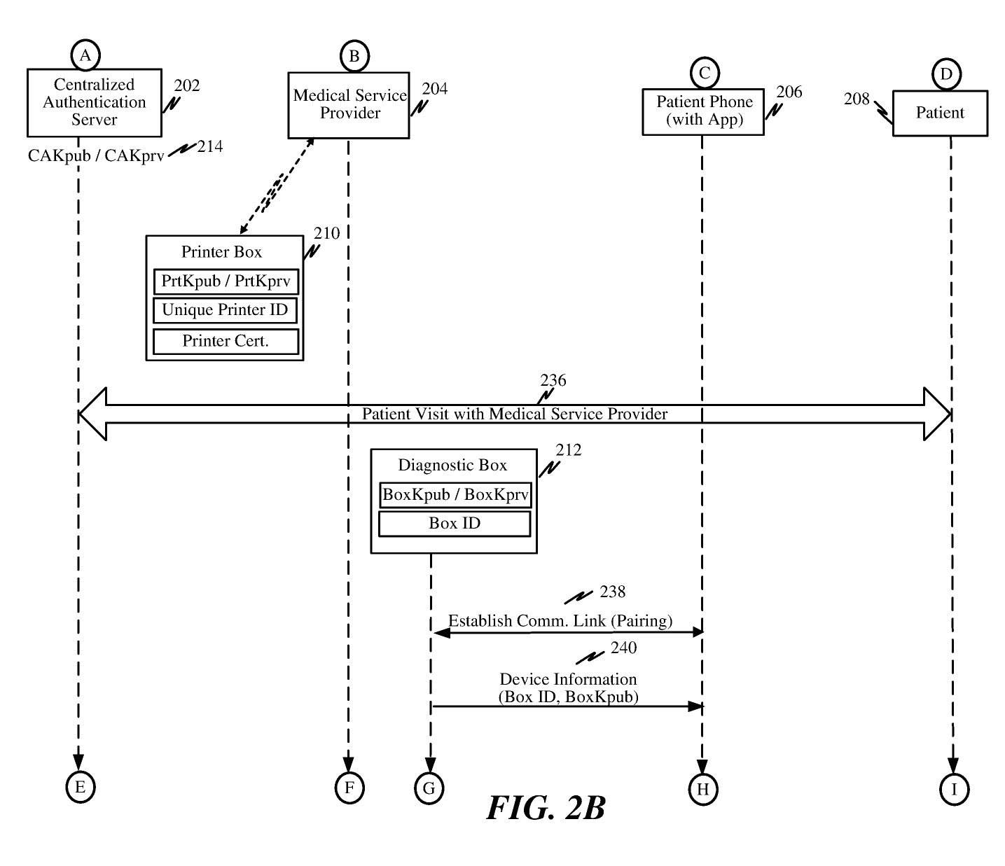

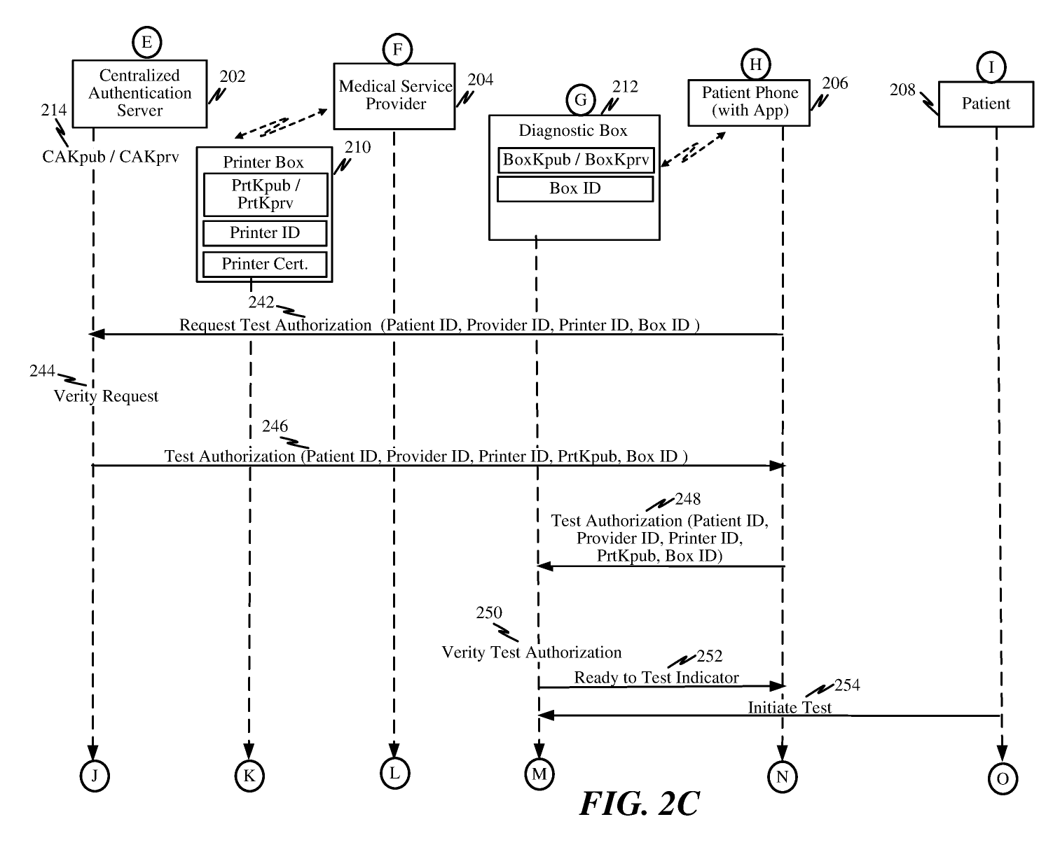

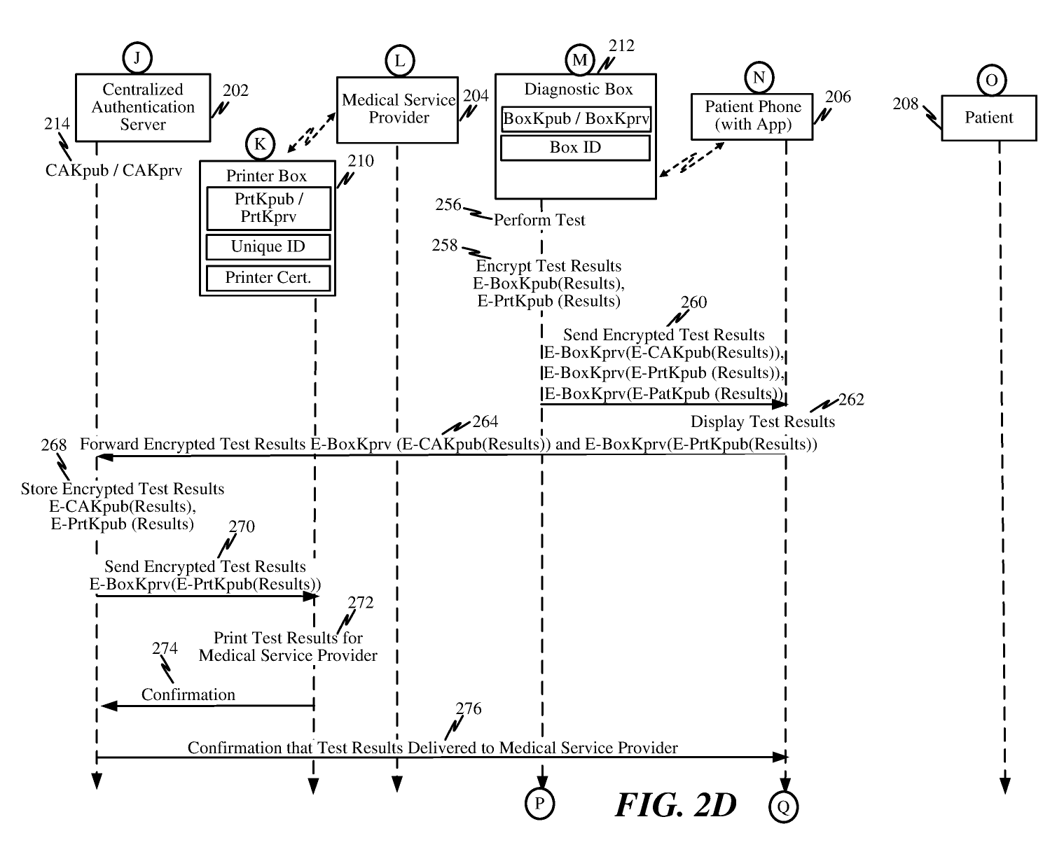

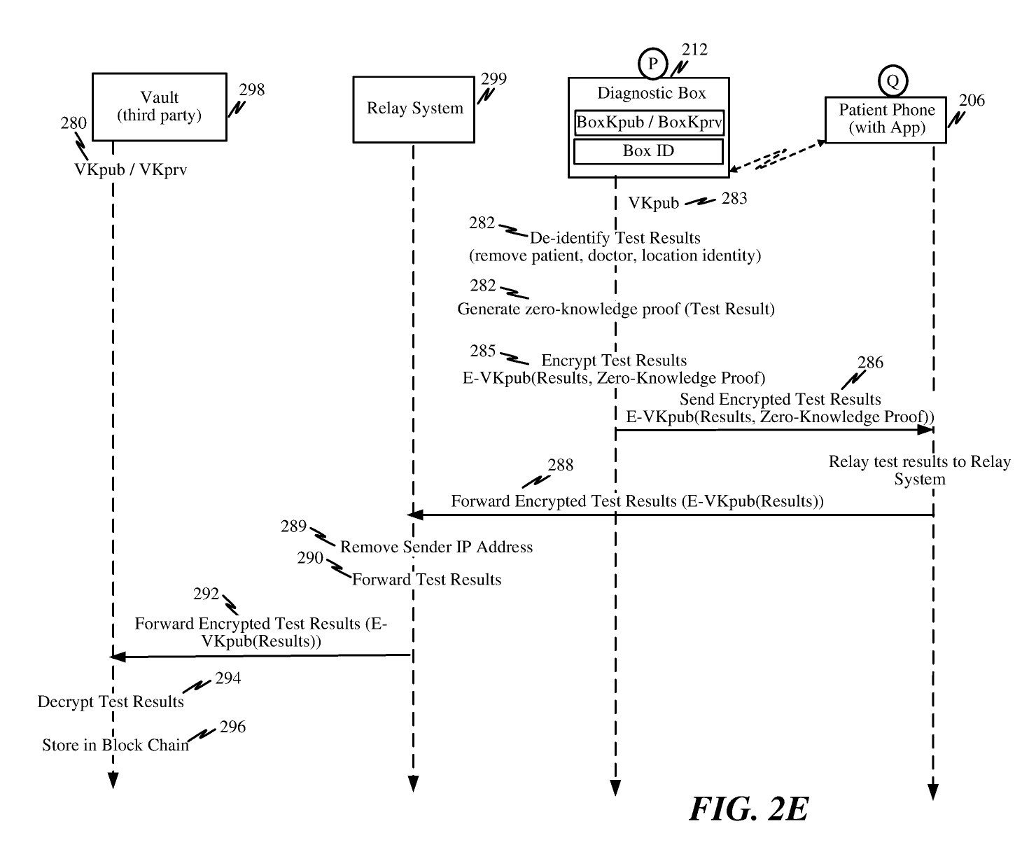

FIG. 2 (comprising FIGS. 2A, 2B, 2C, and 2D) illustrates a second example of a system for using a reusable diagnostics/testing device to test bodily fluids (e.g., blood, to monitor for the presence of a particular drug, etc.) and securely distribute test results to patients and/or medical service providers. Such a re-usable testing device might be installed at, for example, a clinic at a pharmacy, and operated by non-specialized medical professional such as a nurse practitioner or phlebotomist.

This system comprises a centralized authentication server 202 (e.g., centralized service), a medical service provider 204, a printer box/device 210, a patient 208, a patient phone 206 (e.g., patient communication device), and/or a reusable diagnostic/testing box/device 212. A certificate authority public CAKpub and private CAKpry key pair may be defined. The public key CAKpub may be shared with other parties so as to secure (e.g., digitally sign communications to the centralized authentication server).

During an enrollment of provider stage 216, the medical service provider 204 may request to enroll 218 with the centralized authentication server 202 (e.g., or the centralized service). In response to the enrollment request 218, the centralized authentication server 202 may obtain/generate a patient-specific public PrtKpub and private PrtKpry key pair and associates it with a printer box/device 210 that is sent to the medical service provider 204. The printer box/device 210 may also have a unique printer identifier and/or a printer certificate. The printer public key PrtKpub, the unique printer identifier, and/or a printer certificate may be securely sent 222 within the printer box/device 210.

At a patient registration stage 224, the patient 208 may load a software application 226 into the patient phone 206 (e.g., or other personal communication device such as a tablet). Upon attempting an initial registration the software application may generate or obtain a patient public/private key pair PatKpub/PatKpry 227. The software application then sends a patient registration request 228 to the centralized authentication server 202, where the patient registration request 228 may include patient information (e.g., name, address, a patient identifier, etc.) and the patient public key PatKpub. The centralized authentication server 202 may then obtain patient certificates 230 and sends a patient registration confirmation 232, along with the patient certificates 230, to the patient phone 206.

During a patient enrollment stage 235, the patient 208 may visit the medical service provider 204 and performs a patient enrollment procedure in which the patient's phone 206 establishes a communication link 237 with the printer box 210. The patient's phone 206 then sends a patient enrollment request 239 to the printer box 210. In reply, the patient's phone 206 (or software application therein) receives the printer identifier (Printer ID) and/or the printer public key PrtKpub 241, which it can subsequently use to send test results from the diagnostic box 212 to the printer box 210.

During a patient visit stage 236, the patient 208 may periodically visit to the medical service provider 204 (e.g., a primary care facility, a local pharmacy, etc.), who will have the reusable diagnostic box 212. The patient 208 may start the application on the patient phone 206 seek to start the test. Around the same time, the diagnostic box 212 may be turned on. The patient phone 206 and the diagnostic box 212 may establish a communication link 238 (e.g., automatically pair using Bluetooth). No link security is actually needed at this point, because the security may be done at a higher protocol layer.

The diagnostic box 212 may be pre-configured with a unique box identifier, and a box public/private key pair that may also be known to the centralized authentication server 202. The diagnostic box 212 will not perform tests without authorization, which might simplify billing and/or prevent use of the diagnostic box without authorization from the centralized authentication server). Consequently, once the communication link is established 238, the patient phone 206 may receive device information 240 from the diagnostic box 212, including a unique box identifier and a diagnostic box public key BoxKpub. The patient phone 206 may then send a test authorization request 242 to the centralized authentication server 202 (using a secure/authenticated connection, e.g., using CAKpub), the test authorization request 242 including the patient identifier, a medical provider identifier, a printer identifier, and a box identifier. In turn, the centralized authentication server 202 may verify the request 244, and provide a test authorization 246 to the patient phone 206. Upon receiving the test authorization 246, the patient phone 206 forwards it 248 to the diagnostic box 212. The diagnostic box 212 verifies the test authorization 250 and sends an indicator 252 that it is ready to perform the test.

The patient 208 may then initiate the test 254 using the diagnostic box 212. The diagnostic box 212 performs the test 256. When the test result is available, two [optionally three] copies of the result are encrypted 258 by the diagnostic box 212 and sent 260 to the patient phone 206. In one example, the test results may be encrypted using the certificate authority public key CAKpub to produce a first encrypted result E-CAKpub(Results). In another example, the test results may also be encrypted using the printer box public key PrtKpub to produce a second encrypted result E-PrtKpub(Results).

In some implementations, the test results may be further signed using the diagnostic box private key BoxKpry so that a receiving party/entity can be certain that the test was performed by the diagnostic box 212 (i.e., by using the diagnostic box public key BoxKpub to verify).

The first and second encrypted results E-BoxKprv(E-CAKpub(Results)) and E-BoxKprv(E-PrtKpub(Results)) are sent 260 to the patient phone 206. According to an optional aspect, the test results may also be sent to the patient phone 206, for instance, by encrypting the results using the patient public key PatKpub and then diagnostic box private key BoxKpry to obtain a third encrypted result E-BoxKprv(E-PatKpub(Results)). The results may be displayed 262 by the software application on the patient device 206.

In some implementations, the test may be performed by an intermediate or third part (e.g., pharmacy, etc.) which may also be enrolled (e.g., to obtain a public/private key pair). In such case, the intermediate or third party may also receive and view the results, which are further encrypted using a key for the intermediate or third party.

The results first and second encrypted results E-BoxKprv(E-CAKpub(Results)) and E-BoxKprv(E-PrtKpub(Results)) are both forwarded 264 to the centralized authentication server 202 by the patient phone 206. The centralized authentication server 202 may store 268 both encrypted results for backup and potential legal purposes. The second encrypted result E-BoxKprv(E-PrtKpub(Results)) may be delivered/sent 270 to the printer box 210 from where the medical service provider 204 can print 272 and/or store them in a medical records system. The printer box 210 can verify the signature on the result to confirm that the test was performed by a specific diagnostic box. That is, the printer box 210 may use the diagnostic box public key BoxKpub and the printer box private key PrtKpry to decrypt the encrypted results.

The printer box 210 may send a confirmation reply 274 upon receipt of the encrypted test results E-BoxKprv(E-PrtKpub(Results)). In response, the centralized authentication server 202 may send a confirmation 276, to the patient phone 206, that the test results have been delivered to the medical service provider 202. This informs the patient 208 that medical service provider 204 has received the test results.

It is contemplated that the various steps, stages, and/or operations described herein may be combined, rearranged, and/or modified without departing from the novel aspects described.

In addition to the aspects already described, the security infrastructure described herein (e.g., public/private key pairs) may be used for other communications between entities and/or devices in the presented infrastructure. For example, a medical service provider may utilize the communication and/or security mechanism (e.g., public/private key pair) to communicate with a patient's phone to send a message requesting the patient to come in for a visit, while having authenticated proof that the message was delivered to the patient's phone.

### Anonymously Distributing Test Results From Reusable Testing/Diagnostic Device/Box

FIG. 2 (specifically FIG. 2E) further illustrates an exemplary approach for anonymously distributing the test results to third parties. There are about 13 billion lab tests performed annually in the US. Besides the obvious use of these results to diagnose/treat patients, this represents a wealth of analytical data that in aggregate could be used to research and develop new treatments, disease prevention strategies, and health care policies. Such analytical data must be de-identified (e.g., anonymized), so that is not traceable back to individual patients, to satisfy various government regulations such as HIPAA.

In the context of the system mentioned above, the lab test device does know the identity and medical details of the patient for whom the test is authorized. As well as returning the test results to the patient's doctor, the device could also de-identify the data and forward the anonymized results to a data repository (e.g., vault) for analysis. However, to preserve privacy the reporting device must also be anonymous, or it will be possible, in some cases, to correlate test devices with patients. For example, a home test device would uniquely identify the user, and a test device at a pharmacy clinic, used to measure the level of a drug to treat an uncommon disease, might similarly reveal the identity of the only patient in that area with that particular disease.

On the other hand, if the device reporting the results is anonymous, the analytics repository (e.g., vault) cannot protect itself from bogus submissions, making the data unreliable.

According to one aspect, once the diagnostic box 212 (e.g., lab/test device) performs a successful test 256 (FIG. 2D), it may de-identify the test result 282, removing any information that might identify the patient (e.g., patient name, patient identifier, doctor information, etc.). This leaves the test result itself and perhaps generic data about the age range, test date, geographical area or other non-specific information.

A zero-knowledge proof may then be calculated 284 linking the test result and a proof that the diagnostic box 212 possesses a secret key (e.g., BoxKprv) registered within the centralized authentication server 202. This is the same test diagnostic box secret key that is used to sign the data going back to the medical service provider 204. While the zero-knowledge proof does not reveal the secret key (or even the public key), it does prove that the diagnostic box/device has been provisioned with some secret key known within the system, which can be taken as evidence that it is not providing bogus data. The zero-knowledge proof 282 may allow a recipient (e.g., vault 298) to verify that the test results are not bogus data.

In zero-knowledge proofs, the prover can prove to the verifier that it possesses some secret without revealing any information about the secret to the verifier. In this case, the diagnostic box 212 will prove to the vault 298 that it knows the secret key unique to the diagnostic box, without revealing to the vault 298 either which secret key it knows or which device that key is associated with. This proof, combined with the test results, may serve to convince the vault 298 that the test results are not bogus. A more recent development in this area is the “zkSNARK”. The acronym zk-SNARK stands for “Zero-Knowledge Succinct Non-Interactive Argument of Knowledge,” and refers to a proof construction where one can prove possession of certain information, e.g. a secret key, without revealing that information, and without any interaction between the prover and verifier. See e.g. https://z.cash/technology/zksnarks.html. zk-SNARCs have been used to implement the z-cash cryptocurrency.

The test results and zero-knowledge proof may be encrypted using a previously obtained vault (e.g., third party) public key VKpub 285. Note that the vault public key VKpub 283 may be part of a cryptographic key pair (i.e., public key VKpub, private key VKprv) 280 associated with the vault 298 (e.g., third party) to which the anonymous test results are to be sent.

The encrypted test results and zero-knowledge proof E-VKpub(Results, Zero-Knowledge Proof)) are then sent 286 to the patient phone 206. The patient phone 206 may then relay or forward 288 the encrypted test results and zero-knowledge proof E-VKpub(Results, Zero-Knowledge Proof)) to a relay system 299.

The relay system 299 removes an originating network address or device address (e.g., IP address), and forwards the encrypted content to the vault 298. The vault 298 decrypts 294 the test results and zero-knowledge proof using its private key VKprv, and stores it 296 (along with the proof) in either/both an internal database or a public blockchain. The accumulated test results can be provided safely to researchers and health organizations. A more sophisticated anonymizing forwarding system such as The Onion Router (TOR) could instead be used.

Since no eavesdropper can see the test results because they are encrypted during transmission, they will be unable to trace any particular result back to the originating test device. Any party that receives the decrypted test results will be able to verify the zero-knowledge proof, thus knowing that this is a valid test result and not fraudulent data.

Note that the method illustrated in FIG. 2E may be performed independent of the methods/steps illustrated in FIGS. 2A, 2B, 2C, and/or 2D. Moreover, the method illustrated in FIG. 2E may be performed with other testing methods (e.g., FIG. 1) to anonymously distribute test results to third parties (e.g., vault).

### Exemplary Diagnostic Device and Method Operational Therein

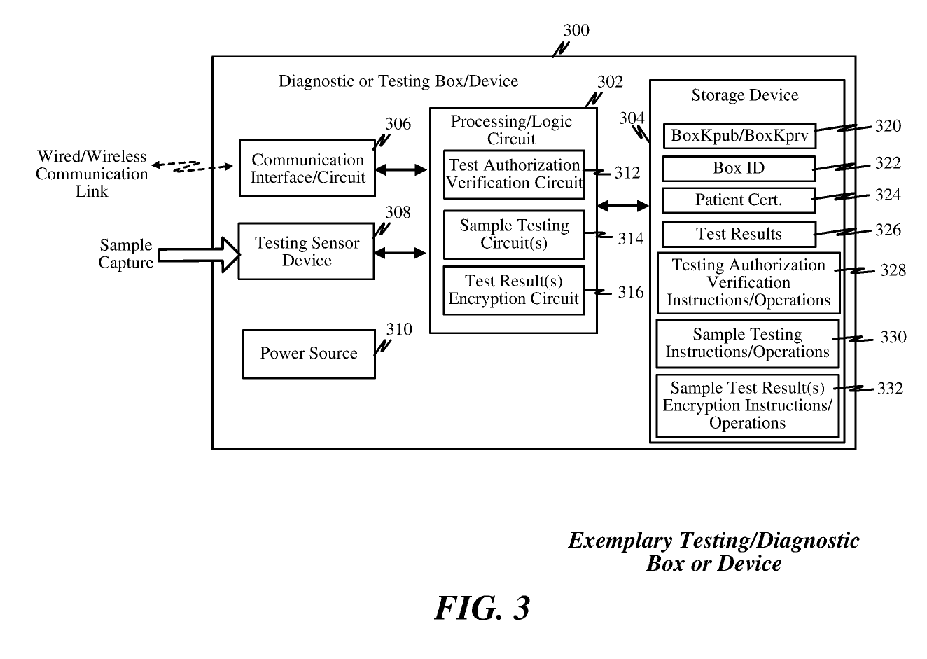

FIG. 3 illustrates an exemplary diagnostic device 300 configured to test one or more bodily fluids or biological samples and securely provide test results. The diagnostic device 300 may perform one or more functions and/or steps described in FIGS. 1 and 2 with relation to the diagnostic box 112 and/or 212. In various configurations, the diagnostic box may be disposable (e.g., one-time-use device) or it may be reusable.

The diagnostic box 302 may include a processing/logic circuit 302 coupled to a storage device 304, a communication interface/circuit 306, and/or a testing sensor device 308. A power source 310 maybe internal or external to the device 300, such as a battery, power cell, etc., and may serve to power the processing/logic circuit 302, storage device 304, communication interface 306, and/or sensor device 308. The communication interface/circuit 306 may be configured to transmit information to/from the diagnostic device 300. The processing circuit 302 may be configured to: (a) establish a communication link with a mobile communication device, where the mobile communication device is associated with a unique patient identifier; (b) receive a test request from the mobile communication device including the patient identifier; (c) verify whether the requested test can be performed based, at least partially, on the patient identifier and/or box identifier; (d) perform the requested test, if the patient identifier is verified, to obtain test results; (e) encrypt the test results using the first private key and a first authorized receiver public key to obtain a first encrypted result; (f) encrypt the test results using the first private key and a second authorized receiver public key to obtain a second encrypted result; and/or (g) send the first encrypted result and second encrypted result to the mobile communication device.

The diagnostic device 300 may be provisioned, pre-loaded, or pre-configured with a first private key BoxKpry and a first public key BoxKpub cryptographic pair 320, and a unique box identifier 322 stored in the storage device 304. The cryptographic key pair 320 may be used to encrypt or cryptographically secure test results sent from the diagnostic box 300. The box identifier 322 may serve to restrict usage of the diagnostic box 300 to an approved user/patient (e.g., a user/patient that is able to provide the same box identifier). Additionally, the box identifier 322 may serve to associate test results with the diagnostic box 300.

A test authorization verification circuit/module 312 may serve to ascertain whether a user/patient requesting a test is authorized to do so on the diagnostic device 300.

The sensor device 308 may serve to capture, receive, extract, or obtain a fluid sample (e.g., blood, saliva, sweat, etc.) and/or other biological sample (e.g., hair, skin, etc.) from a user or patient. A sample testing circuit 314 may then perform one or more tests on the sample and produces or obtains test results. The test results 326 may stored in the storage device 304.

The test results may be encrypted for transmission via the communication interface/circuit 306. For instance, the diagnostic box private key and, optionally, one or more intended recipient public keys may be used to encrypt and/or secure the test results prior to transmission and/or for storage (e.g., FIG. 1, steps 164 and 168, FIG. 2, steps 258 and 260).

The storage device 304 may also include testing authorization verification instructions/operations 328, sample testing instructions/operations 330, and/or sample test results encryption instructions/operations 332 that may be executed by the processing circuit 302.

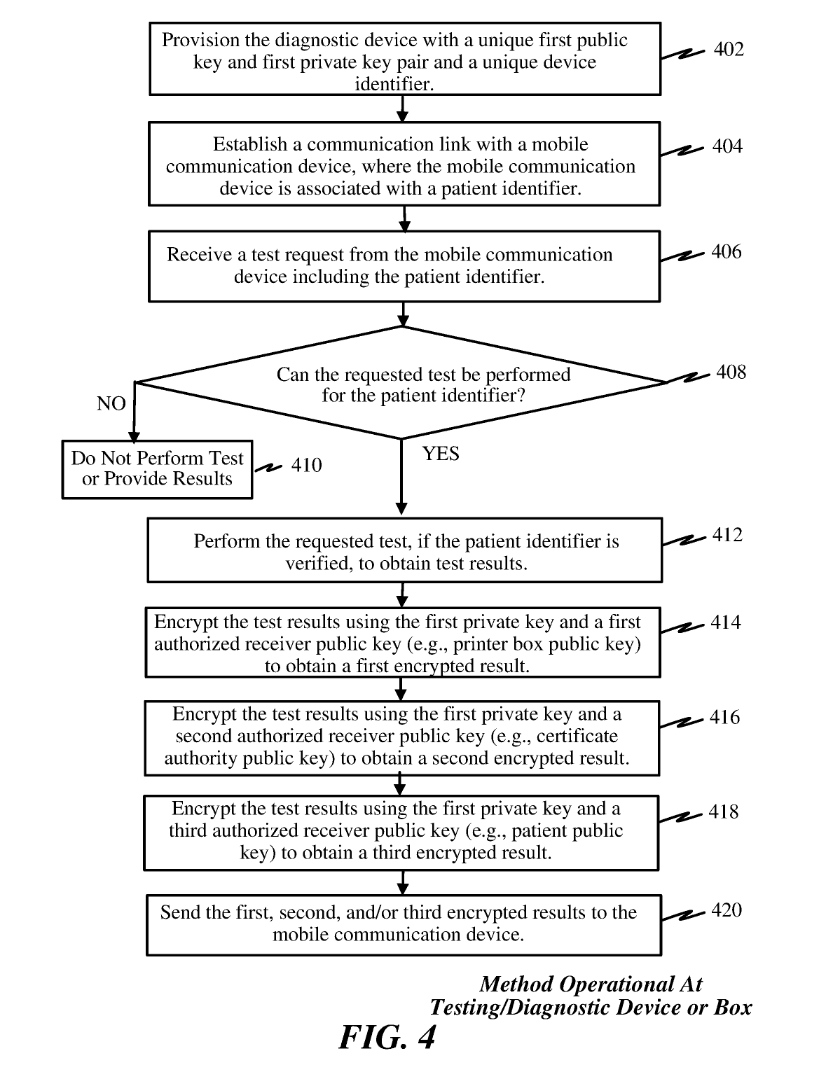

FIG. 4 illustrates a method operational in a diagnostic device to securely share test results. The diagnostic device may be provisioned with a unique first public key and first private key pair and a unique device identifier for the diagnostic device 402. A communication link may be established with a mobile communication device, where the mobile communication device is associated with a unique patient identifier 404. A test request may then be receive from the mobile communication device including the patient identifier 406. The diagnostic device may verify whether the requested test can be performed based, at least partially, on the patient identifier 408. The requested test is performed, if the patient identifier is verified, to obtain test results 412. The diagnostic device does not perform the test or provides results if the verification fails. The test results may be encrypted using the first public key and a first authorized receiver public key (e.g., a printer box public key) to obtain a first encrypted result 414. The test results may also be encrypted using the first private key and a second authorized receiver public key (e.g., certificate authority public key) to obtain a second encrypted result 416. Additionally, the test results may also be encrypted using the first private key and a third authorized receiver public key (e.g., patient public key) to obtain a third encrypted result 418. The first encrypted result and second encrypted result are sent to the mobile communication device 420. In various examples, the requested test may be performed: (a) on blood or bodily fluids to detect one or more medical conditions, or (b) to detect a level of a particular drug.

In some implementations, the diagnostic device may be pre-configured with a stored patient identifier, and verifying whether the requested test can be performed includes comparing the stored patient identifier and the patient identifier received with the test request. The diagnostic device may also be pre-configured to work only if the test is requested with the unique patient identifier. According to one aspect, the test request may further include a diagnostic device identifier, and the method further comprises confirming whether the unique device identifier matches the diagnostic device identifier prior to proceeding with the test request. The test request may further include a current date and/or current time that the diagnostic device appends to the test result.

In other implementations, the diagnostic device may be a reusable device. In that case, a test authorization request is sent to an authorization server via the mobile communication device, where the authorization request includes the unique device identifier, and the first public key. A test authorization message may then be received from the authorization server via the mobile communication device if the test authorization request is authorized. The test authorization message may include the second authorized receiver public key and a printer device identifier associated with the second authorized receiver public key.

### Exemplary Printer Device and Method Operational Therein

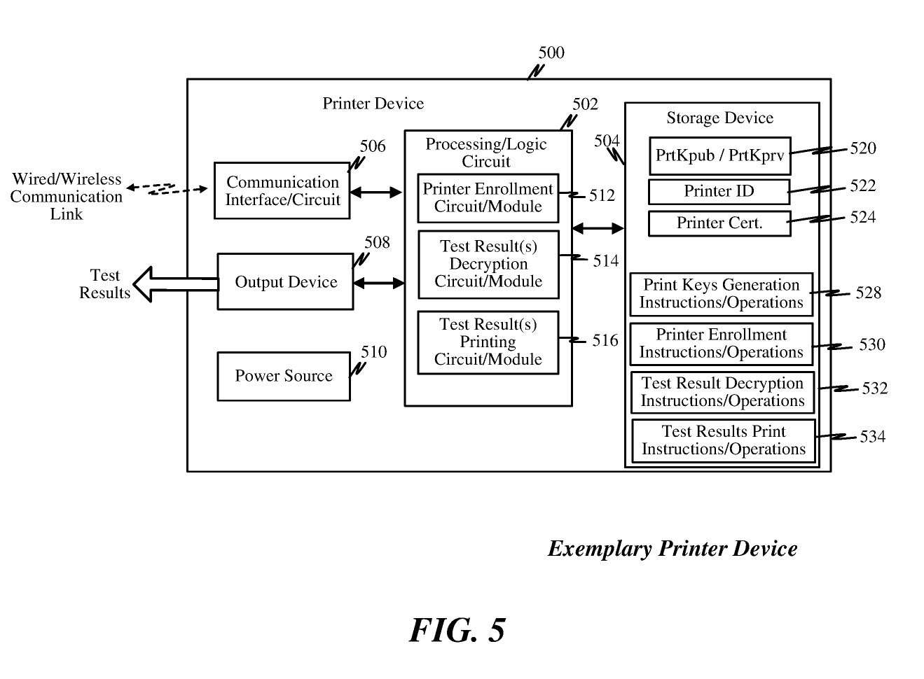

FIG. 5 illustrates an exemplary printer device 500 configured to securely provide test results. The printer device 500 (e.g., a mobile/wireless phone, tablet, etc.) may perform one or more functions and/or steps described in FIGS. 1 and 2 with relation to the printer box 110 and/or 210.

The printer device 500 may include a processing circuit 502 coupled to a storage device 504, a communication interface/circuit 506, and/or an output device 508. A power source 510 maybe internal or external to the device 500, such as a battery, power cell, etc., and may serve to power the processing circuit 502, the storage device 504, the communication interface/circuit 506, and/or the output device 508. The communication interface/circuit 506 may be configured to transmit information to/from the printer device 500.

The printer device 500 may be provisioned or configured with a printer private key PrtKpry and a printer public key PrtKpub cryptographic pair 520, and a unique printer identifier 522 stored in the storage device 504. In one example, a printer keys generation circuit/module 512 may serve to generate the cryptographic key pair 520. For instance, the processing circuit 502 may implement printer key generation instructions/operations 528 to obtain the printer private key PatKpry and a patient public key PatKpub 520.

A printer enrollment circuit/module 514 may serve to enroll or register the printer with an authentication server. For instance, the processing circuit 502 may implement patient enrollment instructions/operations 530 to achieve these functions. A unique printer identifier 522 and a printer certificate(s) 524 may be stored at the storage device 504.

A test result decryption circuit/module 514 may serve to receive encrypted test results from the authentication server and decrypt them using is printer private key and/or a diagnostic device public key. For instance, the processing circuit 502 may implement test result decryption instructions/operations 532 to achieve these functions. Once the received encrypted test results are decrypted, a test results printing circuit/module 516 may be printed to provide the test results via the output device 508. For instance, the processing circuit 502 may implement test results print instructions/operations 534 to achieve these functions.

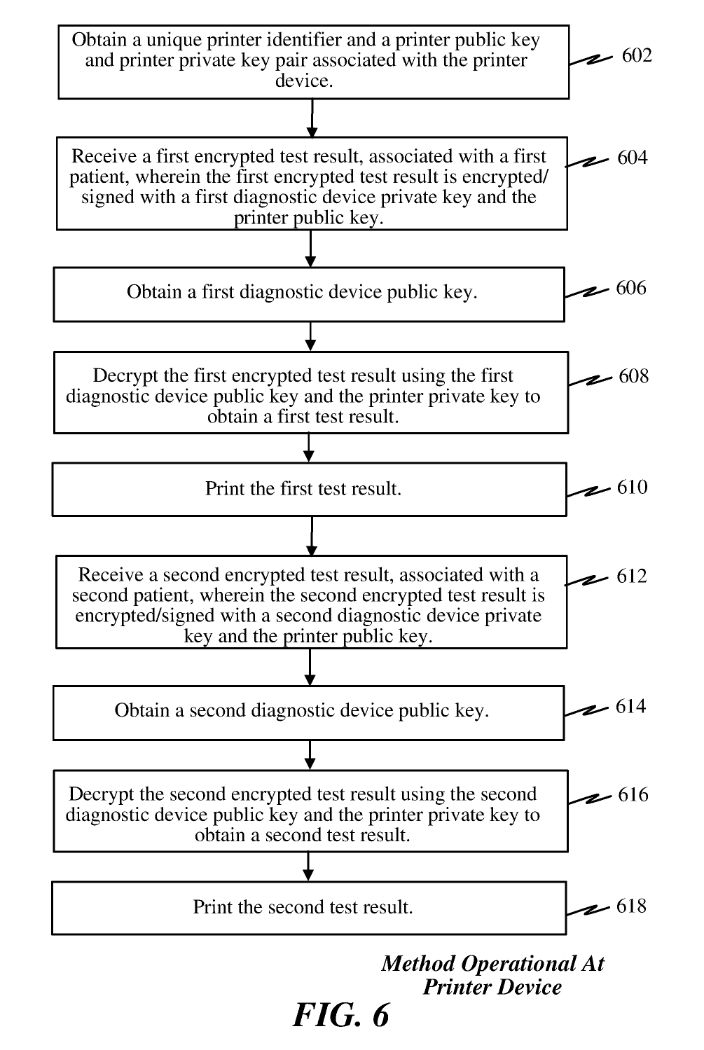

FIG. 6 illustrates a method operational by a printer device to facilitate secure delivery of test results. A unique printer identifier and a printer public key and printer private key pair are obtained which are associated with a printer device 602. A first encrypted test result, associated with a first patient, is received that is encrypted/signed with a first diagnostic device private key and the printer public key 604. The printer may also obtain a first diagnostic device public key 606. The printer device is then able to decrypt the first encrypted test result using the first diagnostic device public key and the printer private key to obtain a first test result 608. The first test result is then printed or otherwise displayed/stored (e.g., as part of medical records) 610.

A second encrypted test result, associated with a second patient, is received that is encrypted/signed with a second diagnostic device private key and the printer public key 612. The printer may also obtain a second diagnostic device public key 614. The printer device is then able to decrypt the second encrypted test result using the second diagnostic device public key and the printer private key to obtain a second test result 616. The second test result is then printed or otherwise displayed/stored (e.g., as part of medical records) 618.

### Exemplary Mobile Communication Device and Method Operational Therein

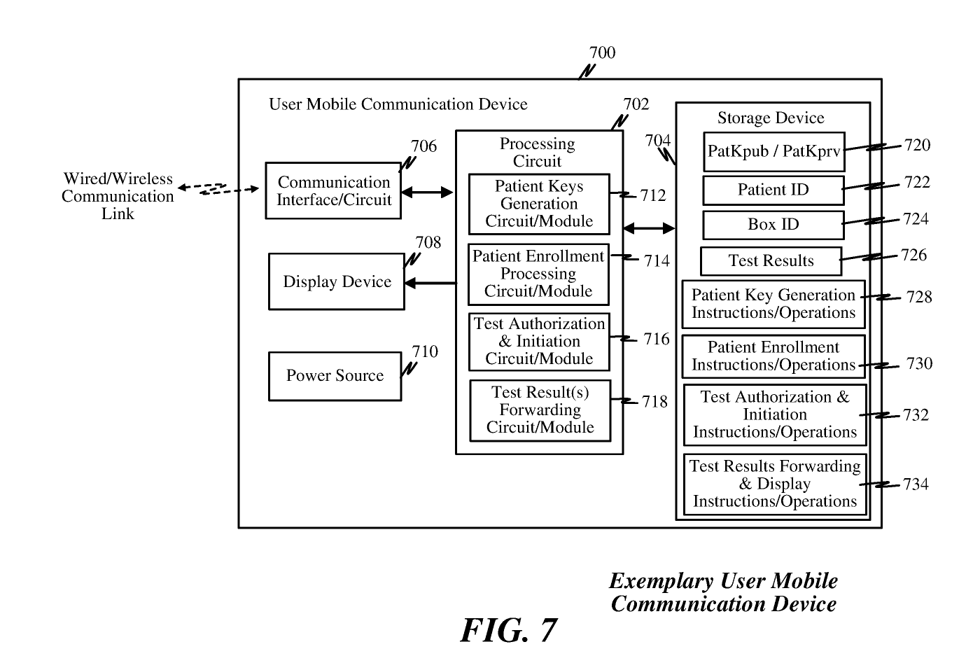

FIG. 7 illustrates an exemplary mobile communication device 700 configured to test one or more bodily fluids or biological samples and securely provide test results. The mobile communication device 700 (e.g., a mobile/wireless phone, tablet, etc.) may perform one or more functions and/or steps described in FIGS. 1 and 2 with relation to the patient phone 106 and/or 206.

The mobile communication device 700 may include a processing circuit 702 coupled to a storage device 704, a communication interface/circuit 706, and/or a display/output device 708. A power source 710 maybe internal or external to the device 700, such as a battery, power cell, etc., and may serve to power the processing circuit 702, the storage device 704, the communication interface 706, and/or the display device 708. The communication interface/circuit 706 may be configured to transmit information to/from the mobile communication device 700.

The mobile communication device 700 may be provisioned or configured with a patient private key PatKpry and a patient public key PatKpub cryptographic pair 720, and a unique patient identifier 722 stored in the storage device 704. In one example, a patient keys generation circuit/module 712 may serve to generate the cryptographic key pair 720. For instance, the processing circuit 702 may implement patient key generation instructions/operations 728 to obtain the patient private key PatKpry and a patient public key PatKpub 720.

A patient enrollment circuit/module 714 may serve to enroll or register the user/patient with an authentication server. For instance, the processing circuit 702 may implement patient enrollment instructions/operations 730 to achieve these functions. A patient identifier 722 and a diagnostic box identifier 724 may be stored at the storage device 704.

A test authorization and initiation circuit/module 716 may serve to send a request to a diagnostic device to authorize and initiate a test. For instance, the processing circuit 702 may implement test authorization and initiation instructions/operations 732 to achieve these functions. Note that multiple different tests may be requested in an authorization and initiation message sent from the mobile communication device 700 to a diagnostic device.

A test result forwarding circuit/module 718 may serve to receive encrypted test results from a diagnostic device and forward them to an authentication server. For instance, the processing circuit 702 may implement test result forwarding instructions/operations 734 to achieve these functions. The test results may be stored 726 in the storage device 704. Additionally, the encrypted test results may be decrypted at the mobile user communication device 700 and displays it on the display device 708. Additionally, the storage device 704 may optionally store the encrypted test results 726.

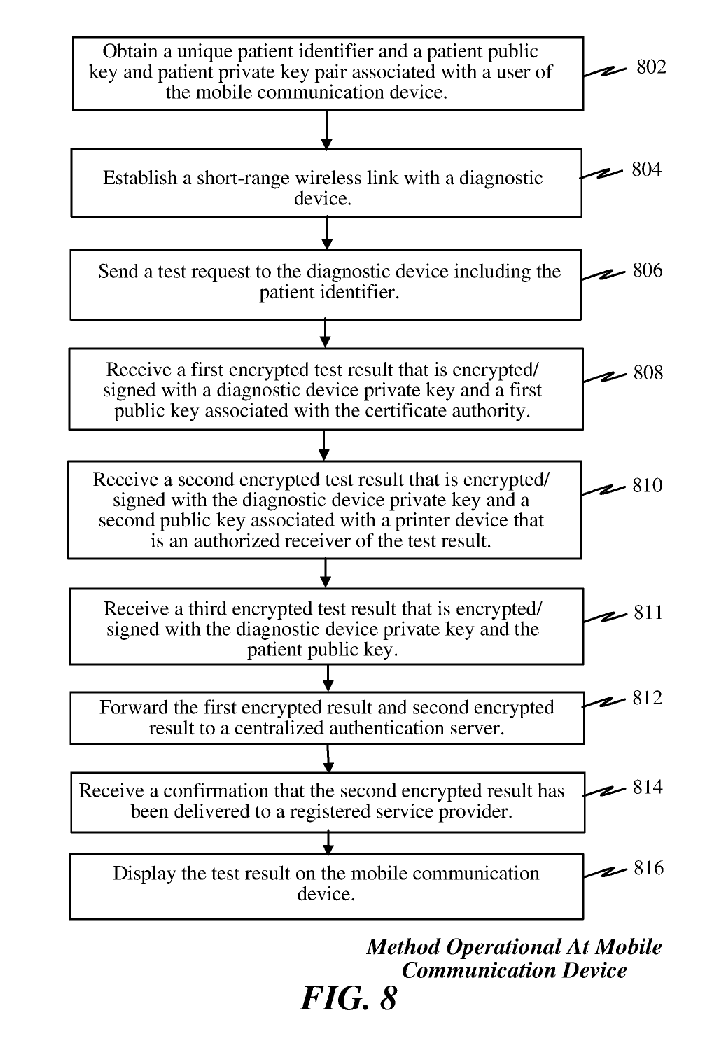

FIG. 8 illustrates a method operational by a mobile communication device to facilitate performing a test with a diagnostic device and securely share test results. A unique patient identifier and a patient public key and patient private key pair are obtained which are associated with a user of the mobile communication device and/or user (patient) of the communication device 802. A short-range wireless link is established with a diagnostic device 804. A test request is sent to the diagnostic device including the patient identifier and/or the patient public key 806. A first encrypted test result is received that is encrypted with a diagnostic device private key and a first private key associated with a certificate authority 808. A second encrypted test result is also received that is encrypted with the diagnostic device private key and a second public key associated with a printer device that is an authorized receiver of the test result 810. A third encrypted test result may also be received that is encrypted with the diagnostic device private key and the patient public key 811. The first, second, and/or third encrypted results may be forwarded to a centralized authentication server 812. A confirmation may be received that the second encrypted result has been delivered to a registered service provider 814. The test result may also be displayed on the mobile communication device 816.

The test request may include the printer device identifier. The requested test may be performed on blood or bodily fluids to detect one or more medical conditions. For instance, the requested test may be performed to detect a level of a particular drug.

In other implementations, one or more of the steps and/or methods described and/or illustrated herein may be implemented as one or more instructions stored in a non-transitory processor-readable storage medium. These instructions may be executed by at least one processing circuit to cause the steps and methods to be implemented.

One or more of the components, steps, features, and/or functions illustrated in the Figures may be rearranged and/or combined into a single component, step, feature or function or embodied in several components, steps, or functions. Additional elements, components, steps, and/or functions may also be added without departing from the invention. The apparatus, devices, and/or components illustrated in the Figures may be configured to perform one or more of the methods, features, or steps described in the Figures. The algorithms described herein may also be efficiently implemented in software and/or embedded in hardware.

Also, it is noted that the aspects of the present disclosure may be described as a process that is depicted as a flowchart, a flow diagram, a structure diagram, or a block diagram. Although a flowchart may describe the operations as a sequential process, many of the operations can be performed in parallel or concurrently. In addition, the order of the operations may be re-arranged. A process is terminated when its operations are completed. A process may correspond to a method, a function, a procedure, a subroutine, a subprogram, etc. When a process corresponds to a function, its termination corresponds to a return of the function to the calling function or the main function.

Moreover, a storage medium may represent one or more devices for storing data, including read-only memory (ROM), random access memory (RAM), magnetic disk storage mediums, optical storage mediums, flash memory devices and/or other machine-readable mediums and, processor-readable mediums, and/or computer-readable mediums for storing information. The terms “machine-readable medium”, “computer-readable medium”, and/or “processor-readable medium” may include, but are not limited to non-transitory mediums such as portable or fixed storage devices, optical storage devices, and various other mediums capable of storing, containing or carrying instruction(s) and/or data. Thus, the various methods described herein may be fully or partially implemented by instructions and/or data that may be stored in a “machine-readable medium”, “computer-readable medium”, and/or “processor-readable medium” and executed by one or more processors, machines and/or devices.

Furthermore, aspects of the disclosure may be implemented by hardware, software, firmware, middleware, microcode, or any combination thereof. When implemented in software, firmware, middleware or microcode, the program code or code segments to perform the necessary tasks may be stored in a machine-readable medium such as a storage medium or other storage(s). A processor may perform the necessary tasks. A code segment may represent a procedure, a function, a subprogram, a program, a routine, a subroutine, a module, a software package, a class, or any combination of instructions, data structures, or program statements. A code segment may be coupled to another code segment or a hardware circuit by passing and/or receiving information, data, arguments, parameters, or memory contents. Information, arguments, parameters, data, etc. may be passed, forwarded, or transmitted via any suitable means including memory sharing, message passing, token passing, network transmission, etc.

The various illustrative logical blocks, modules, circuits, elements, and/or components described in connection with the examples disclosed herein may be implemented or performed with a general purpose processor, a digital signal processor (DSP), an application specific integrated circuit (ASIC), a field programmable gate array (FPGA) or other programmable logic component, discrete gate or transistor logic, discrete hardware components, or any combination thereof designed to perform the functions described herein. A general purpose processor may be a microprocessor, but in the alternative, the processor may be any conventional processor, controller, microcontroller, or state machine. A processor may also be implemented as a combination of computing components, e.g., a combination of a DSP and a microprocessor, a number of microprocessors, one or more microprocessors in conjunction with a DSP core, or any other such configuration.

The methods or algorithms described in connection with the examples disclosed herein may be embodied directly in hardware, in a software module executable by a processor, or in a combination of both, in the form of processing unit, programming instructions, or other directions, and may be contained in a single device or distributed across multiple devices. A software module may reside in RAM memory, flash memory, ROM memory, EPROM memory, EEPROM memory, registers, hard disk, a removable disk, a CD-ROM, or any other form of storage medium known in the art. A storage medium may be coupled to the processor such that the processor can read information from, and write information to, the storage medium. In the alternative, the storage medium may be integral to the processor.

Those of skill in the art would further appreciate that the various illustrative logical blocks, modules, circuits, and algorithm steps described in connection with the aspects disclosed herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system.

The various features of the invention described herein can be implemented in different systems without departing from the invention. It should be noted that the foregoing aspects of the disclosure are merely examples and are not to be construed as limiting the invention. The description of the aspects of the present disclosure is intended to be illustrative, and not to limit the scope of the claims. As such, the present teachings can be readily applied to other types of apparatuses and many alternatives, modifications, and variations will be apparent to those skilled in the art.

What is claimed is:

1. A method operational at a diagnostic device, comprising:
provisioning the diagnostic device with a unique first public key and first private key pair and a unique device identifier for the diagnostic device;
establishing a communication link with a mobile communication device, where the mobile communication device is associated with a unique patient identifier;
receiving a test request from the mobile communication device including the patient identifier;
verifying whether the requested test can be performed based, at least partially, on the patient identifier; and
performing, if the patient identifier is verified, the requested test to obtain a test result.

2. The method of claim 1, further comprising:
encrypting and/or signing the test result using the first private key and a first authorized receiver public key to obtain a first encrypted result; and
sending the first encrypted result to the mobile communication device.

3. The method of claim 1, further comprising:
encrypting and/or signing the test result using the first private key and a second authorized receiver public key to obtain a second encrypted result; and
sending the second encrypted result to the mobile communication device.

4. The method of claim 3, wherein the second authorized receiver public key is associated with printer device of an authorized receiver of the test result, and the test request includes a printer device identifier.

5. The method of claim 1, wherein the requested test is:
(a) performed on blood or bodily fluids to detect one or more medical conditions; or
(b) performed to detect a level of a particular drug.

6. The method of claim 1, further comprising:
receiving a patient enrollment request from the mobile communication device prior to receiving the test request; and
providing a printer identifier and printer public key to the mobile communication device.

7. The method of claim 1, wherein the diagnostic device is pre-configured with a stored patient identifier, and verifying whether the requested test can be performed includes comparing the stored patient identifier and the patient identifier received with the test request.

8. The method of claim 1, wherein the diagnostic device is a one-time-use device and is pre-configured to work only if the test is requested with the unique patient identifier.

9. The method of claim 1, wherein the test request further includes a diagnostic device identifier, and the method further comprises:
confirming whether the unique device identifier matches the diagnostic device identifier prior to proceeding with the test request.

10. The method of claim 1, wherein the test request further includes a current date and/or current time that the diagnostic device appends to the test result.

11. The method of claim 1, wherein the diagnostic device is a reusable device and the method further includes:
sending a test authorization request to an authorization server via the mobile communication device, where the authorization request includes the unique device identifier, and the first public key, and
receiving a test authorization message from the authorization server via the mobile communication device if the test authorization request is authorized.

12. The method of claim 11, wherein the test authorization message includes a second public key and a printer device identifier associated with the second public key.

13. A diagnostic device, comprising
a storage device for storing a unique first public key and first private key pair and a unique device identifier for the diagnostic device;
a testing sensor device receiving blood or bodily fluids and providing a test result;
a communication interface for transmitting information to/from the diagnostic device; and
a processing circuit coupled to the storage device, the testing sensor device, and the communication interface, the processing circuit configured to:
establish a communication link with a mobile communication device, where the mobile communication device is associated with a unique patient identifier;
receive a test request from the mobile communication device including the patient identifier;
verify whether the requested test can be performed based, at least partially, on the patient identifier; and
perform, if the patient identifier is verified, the requested test to obtain a test result.

14. The diagnostic device of claim 13, wherein the processing circuit is further configured to:
encrypt and/or sign the test result using the first private key and a first authorized receiver public key to obtain a first encrypted result;
encrypt and/or sign the test result using the first private key and a second authorized receiver public key to obtain a second encrypted result; and
send the first encrypted result and second encrypted result to the mobile communication device.

15. The diagnostic device of claim 13, wherein the diagnostic device is pre-configured with a stored patient identifier, and verifying whether the requested test can be performed includes comparing the stored patient identifier and the patient identifier received with the test request.

16. The diagnostic device of claim 13, wherein the diagnostic device is a one-time-use device and is pre-configured to work only if the test is requested with the unique patient identifier.

17. The diagnostic device of claim 13, wherein the test request further includes a diagnostic device identifier, and the method further comprises:
confirming whether the unique device identifier matches the diagnostic device identifier prior to proceeding with the test request.

18. A non-transitory processor-readable storage medium having one or more instructions which, when executed by at least one processing circuit, causes the at least one processing circuit to:
establish a communication link with a mobile communication device, where the mobile communication device is associated with a unique patient identifier;
receive a test request from the mobile communication device including the patient identifier;
verify whether the requested test can be performed based, at least partially, on the patient identifier; and
perform, if the patient identifier is verified, the requested test to obtain a test result.

19. The non-transitory processor-readable storage medium of claim 20, further having one or more instructions which, when executed by at least one processing circuit, causes the at least one processing circuit to:
encrypt and/or sign the test result using the first private key and a first authorized receiver public key to obtain a first encrypted result;
encrypt and/or sign the test result using the first private key and a second authorized receiver public key to obtain a second encrypted result; and
send the first encrypted result and second encrypted result to the mobile communication device.
